(19) United States
(12) Patent Application Publication
(10) Pub. No.: US 2017/0095178 A1
(43) Pub. Date: Apr. 6, 2017

(54) EMG Home Trainer

(71) Applicants: Marco Schoen, Pocatello, ID (US); Kurt Scott, Blackfoot, ID (US); Feng Lin, Beijing (CN); Sichen Wang, Beijing (CN); Yun Zhang, Beijing (CN); Lin Yang, Beijing (CN)

(72) Inventors: Marco Schoen, Pocatello, ID (US); Kurt Scott, Blackfoot, ID (US); Feng Lin, Beijing (CN); Sichen Wang, Beijing (CN); Yun Zhang, Beijing (CN); Lin Yang, Beijing (CN)

(21) Appl. No.: 15/096,199

(22) Filed: Apr. 11, 2016

Related U.S. Application Data
(60) Provisional application No. 62/145,500, filed on Apr. 9, 2015.

(51) Int. Cl. A61B 5/0492; A61B 5/04; A61B 5/00; H03F 3/45

(52) U.S. Cl. CPC A61B 5/0492; H03F 3/45744; A61B 5/04012; A61B 5/0004; A61B 5/7207; A61B 5/742; A61B 5/486; H03F 2200/261

(57) ABSTRACT

A system for the detection, recording, and analysis of EMG data attained during exercise in real-time is described. The system employs a surface EMG sensor equipped with a Bluetooth or a Wi-Fi module. The sensor transmits sEMG data via Bluetooth or Wi-Fi to a smart device, which processes this data utilizing a preloaded application and informs the user of the efficacy of exercises with key statistics in real-time. A sensor box containing the EMG sensor also contains a pre-amplification circuit, an amplification circuit, a DC-offset circuit, and a signal smoothing and amplifier circuit to maximize the reduction of extraneous noise frequently observed with conventional sEMG sensors. A proprietary mobile device application receives, interprets, records, and displays the EMG data for the user.

### CONTINUITY

This application is a non-provisional application of provisional patent application No. 62/145,500, filed on Apr. 9, 2015, and priority is claimed thereto.

### FIELD OF THE PRESENT INVENTION

The present invention relates generally the technical field of fitness and rehabilitation. More particularly, the present invention is specific to training and rehabbing the human skeletal musculature.

### BACKGROUND OF THE PRESENT INVENTION

Work-out facilities in public and/or private gyms are supported by trainers with particular expertise in achieving a customer's fitness goals. A customer usually trains for achieving a certain fitness goal. The trainer develops corresponding fitness programs and exercise routines. The outcomes of these routines and programs are assessed over time as the customer endures weeks if not months of physical activities. Routines and exercise programs are altered after this longer term feedback has occurred. However, the trainer and customer have little knowledge on the immediate impact of any one specific exercise routine. For example, while performing a particular exercise on a given exercise machine, neither the trainer nor the customer are able to accurately assess the impact of the current activity.

In a similar fashion, athletes, stroke victims, elderly people, and generally people who are going through physical therapy to regain or strengthen certain movements, are left without any immediate feedback on the effectiveness of the current exercise.

Physical signals of the human body can be extracted by sensors and analyzed to evaluate health conditions. A surface EMG sensor is conventionally used to measure activation level of the muscles. Generally, a cathode, anode, electrodes, and a ground electrode are used in surface EMG sensors. However, the signal obtained from the surface of the skin with conventional EMG sensors has a large noise ratio that hinders the performance and accuracy of the EMG sensor. Therefore, a design with higher quality and accuracy is needed.

Thus, there is a need for an accurate sEMG sensor of high quality that is configured for use on an individual during exercise and/or physical therapy. Such a device is preferably designed to minimize the noise ratio experienced from the placement of sensors on the skin, and provides instant feedback pertaining to the exercises performed by the user in real-time.

### SUMMARY OF THE PRESENT INVENTION

The present invention provides a system for monitoring and displaying surface EMG data of a targeted muscle group. The system comprises a stand-alone surface EMG sensor and a communication device (such as a smart phone) with a corresponding application program (app) to process, record, and display the resulting data provided by the present invention.

The surface EMG sensor is placed on the skin near the area of the muscle to be treated, and senses surface EMG potentials when activated. The surface EMG sensor of the present invention is preferably comprised of the following components:
Four (4) prongs on one side of the sensor box. The two middle sensors capture the surface EMG potential from the skin and produce corresponding analogue signals. The two other prongs provide for the ground signals;
An amplification circuit responsible for magnifying and filtering the analogue sEMG signals followed by converting the analog signals to digital signals;
A processing program to get the characteristic output values;
A wireless transmitter terminal, such as a Bluetooth radio, responsible for sending the characteristic output values and in some cases the digital signals to a mobile device.

The amplification circuit of the present invention contains an instrumentation amplifier, a DC offset circuit, and a operational amplifier. Preferably, the instrumentation amplifier employed by the present invention is an AD8220 JFET input, and the operational amplifier is CMOS. The processing program is downloaded in a Bluetooth chip with a microprocessor.

In the preferred embodiment of the present invention, the preferred sampling frequency of the Bluetooth chip is 1000 Hz. Additionally, the wireless transmitter terminal is a Bluetooth radio, however it is envisioned that alternate embodiments of the present invention may employ a conventional Wi-Fi radio. Preferably, the mobile device employed by the present invention is a smart phone or similar smart device.

The mobile device receives the sensed EMG potential and processed signals. A specific proposed pre-loaded application uses the received signals and allows for the user to receive feedback to his/her exercise routine. The application has different modules to allow the user to select what muscle group is being targeted and what kind of exercise is being conducted. The application on the smart Bluetooth and/or Wi-Fi enabled device also allows for statistical measures of the received EMG signal in order to help with providing instantaneous and long term feedback to the user. The real-time feedback provided to the user allows for adjustments to the exercise routine of the user while performing the exercise, as well as for assessing the effectiveness of the chosen exercise routine on the targeted muscle group.

### BRIEF DESCRIPTION OF THE DRAWINGS

The present invention will be better understood with reference to the appended drawing sheets, wherein:

FIG. 1 is a flow chart of the provided system of the surface EMG home trainer.

FIG. 2 is a schematic view of the components of the sEMG sensor.

FIG. 3 is a view of the signal amplification circuit.

FIG. 4 is a view of the signal offset circuit.

FIG. 5 is a view of the signal combination and smoothing circuit.

FIG. 6 is a general view of the system of the present invention in real application.

FIG. 7 is a view of the component of the provided system of the present invention.

FIG. 8 is a diagram of a user actively using the present invention.

### DETAILED DESCRIPTION OF THE PREFERRED EMBODIMENT OF THE PRESENT INVENTION

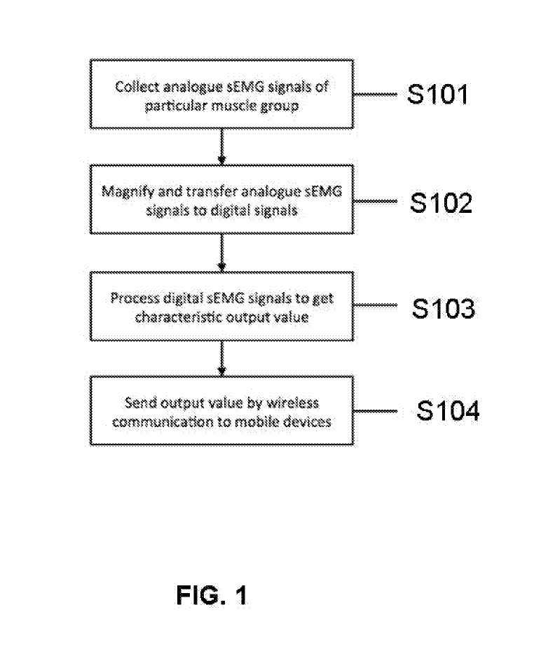

The present invention is a sEMG home training device and system configured for personal use by an individual while exercising or performing physical therapy. As shown in FIG. 1, the invention provided a system of the surface EMG home trainer, which contains a series of steps to achieve the desired result of exercise efficacy and training.

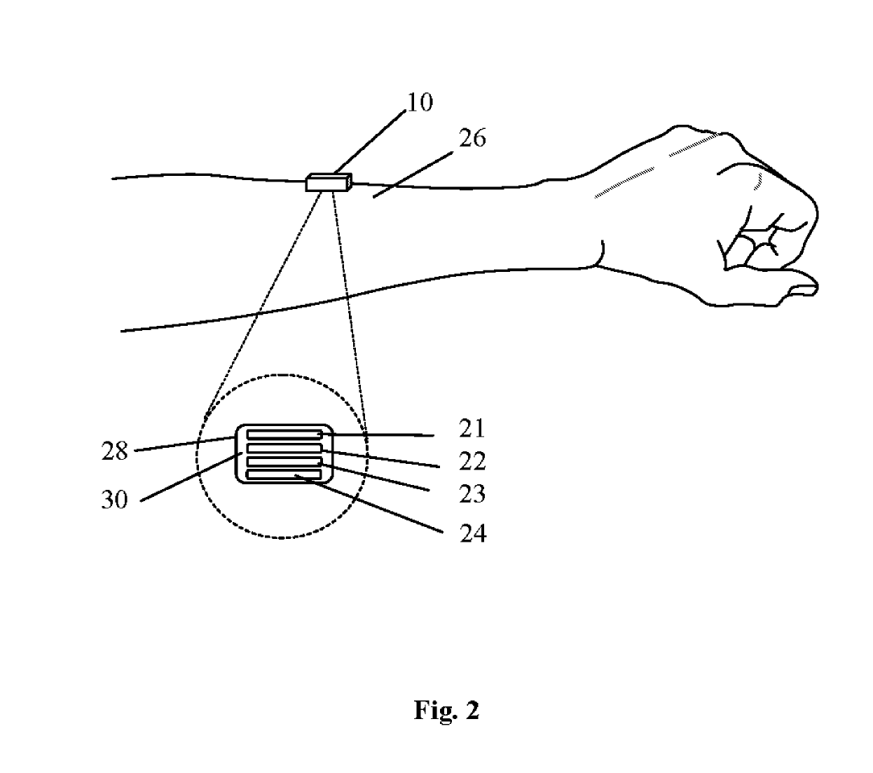

The sensor box (10) of the present invention can be set on any location on human skin. As shown in FIG. 2, the sensor box (10) is set on an arm (26) of the user. Four silver prongs, including a first silver prong (21), a second silver prong (22), a third silver prong (23), and a fourth silver prong (24) are preferably disposed on the bottom of a casing (28) the sensor box (10) to be disposed on the skin. The sensor box can contact with skin by any method. For instance, two-sided sticky paper (27) can be used to attach the sensor box (10) on the skin of the arm (26) of the user, or in other locations on the skin of the user. The present invention has no restriction on the attaching method employed to secure the sensor box (10) to the surface of the user's skin. The process of use of the present invention preferably employs the following four primary steps (S101-S104):

S101, collecting analogue sEMG signals of particular muscle group by sEMG sensor that has four prongs on one side of the sensor box. Middle two prongs, the second silver prong (22) and the third silver prong (23) senses surface EMG potential, and produces analogue signals. The other two prongs, the first silver prong (21) and the fourth silver prong (24) provide ground signals. Preferably, the prongs are coated with silver having 99.99% purity to improve the sampling efficiency, as well as the signal quality, ensuring a reduction of noise.

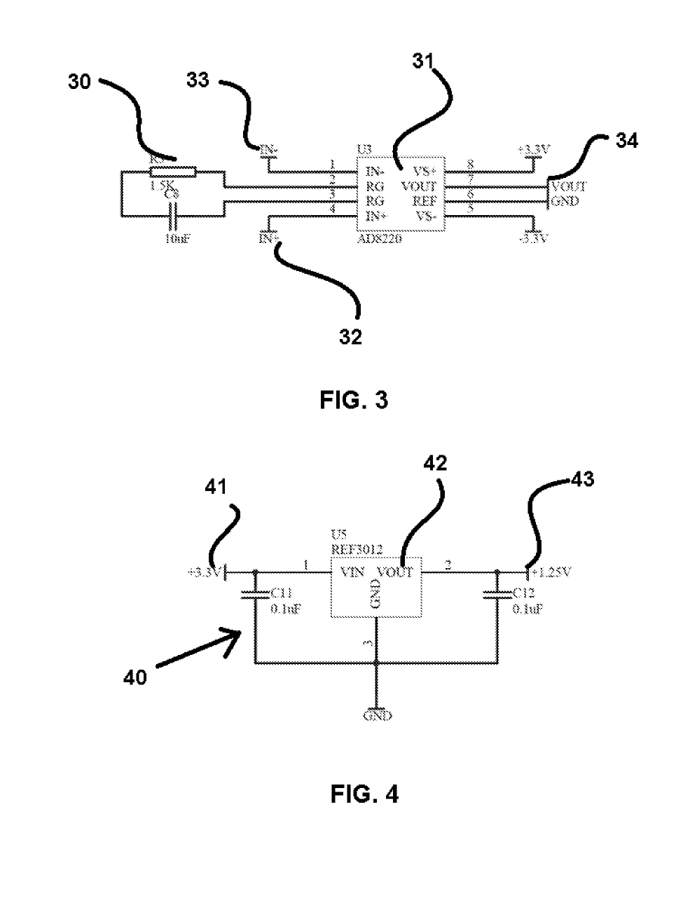

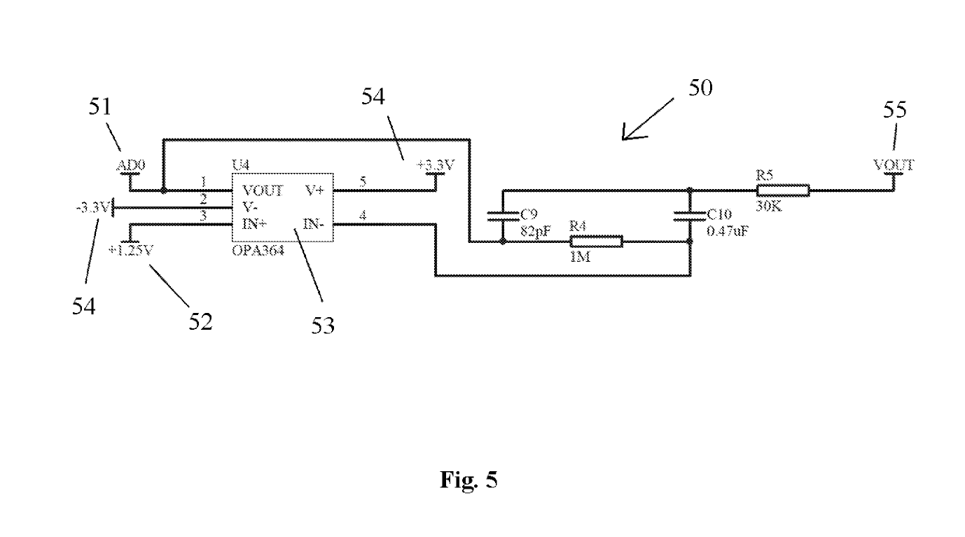

S102, magnifying and transferring analogue sEMG signals to digital signals. Before magnification, filtering is done to the analogue sEMG signals to dele noises. FIG. 3, FIG. 4 and FIG. 5 show the amplification and filtering circuits of the present invention. It should be understood that the present invention does not restrict these circuits, and any circuit that can achieve the signal amplification objective can be used.

Referring now to FIG. 3, the amplification circuit consists of an AD8220 JFET input in station amplifier (31). The input instrumentation amplifier (31) amplifies the differential input of (32) and (33) of the shown circuit using a supply voltage of ±3.3V. The circuit shown in FIG. 4 is the pre-amplifier circuit (30) of the sEMG sensor. The output (34) of the pre-amplifier circuit (30) is then shifted with a DC-offset circuit (40) detailed in FIG. 4.

Referring now to FIG. 4, the dc-offset circuit (40) is given in detail. The DC-offset circuit (40) is powered by a 3.3V power supply (41), and preferably uses a REF3012 voltage reference chip (42). The resulting 1.25V is used to offset the output of the pre-amplifier circuit (30). This offset is accomplished by adding the output (34) of the pre-amplifier circuit (30) to the output (43) of the dc-offset circuit (40).

Referring now to FIG. 5, a signal smoothing and amplifier circuit (50) is given. The signal smoothing and amplifier circuit (50) takes on the output (34) of the pre-amplifier circuit (30) as its input (51), as well as the output (43) of the DC-offset circuit (40) as its input (52). A CMOS operational amplifier (53) is used to combine the output (34) of the pre-amplifier circuit (30) to the output (43) of the dc-offset circuit (40). The CMOS operational amplifier (53) is power by a ±3.3V power supply 54. The resulting output (55) of the amplifier circuit (50) smoothed and amplified.

S103, processing the digital sEMG signals to get characteristic output values. This step is preferably accomplished by using a Bluetooth chip equipped with a microprocessor. It should be understood that the present invention does not restrict the amplification circuits, and that any circuit that can achieve the signal amplification objective can be used. The sampling frequency is preferably 1000 Hz in order to cover the sEMG frequency bandwidth but also different frequencies can be used.

S104, sending the output value by wireless communication to mobile devices. The wireless communication can be any wireless techniques and this invention has no restriction on the range or type of wireless transmission employed. Preferably, this invention uses Bluetooth as the transmitter terminal, and smart phone as the mobile device. A Bluetooth radio is preferably disposed within the smart phone, as well as in the sensor box (10) of the present invention.

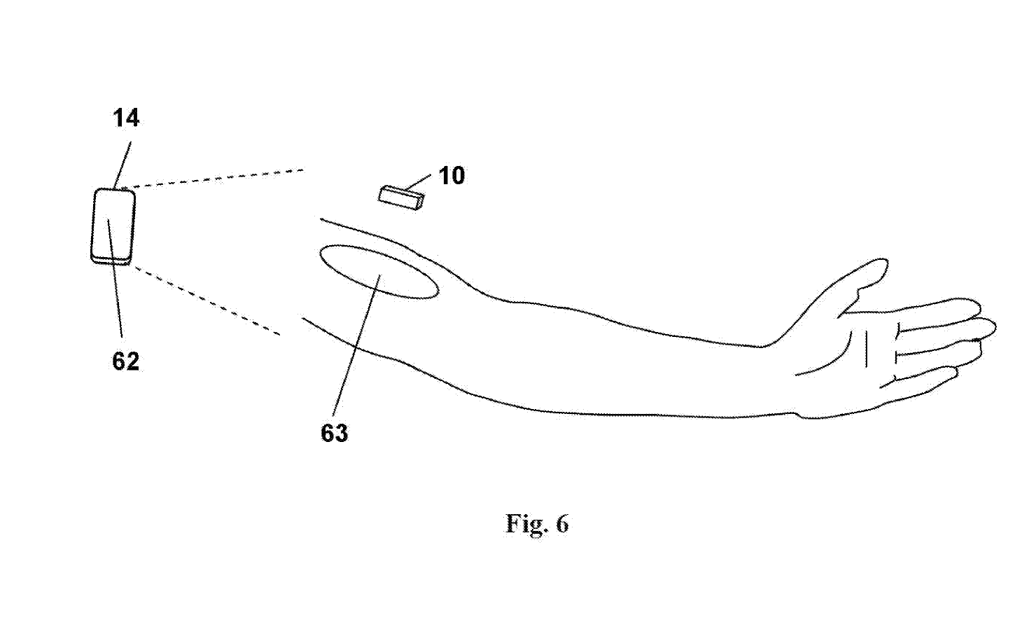

A specific proposed pre-loaded application (62) on the mobile device uses the processed EMG signal, and allows for the user to receive feedback pertaining to his/her exercise routine. As seen in FIG. 6, the application (62) is preferably preloaded on to the mobile device (14). Users can first Choose the exercise or the corresponding muscle as shown in FIG. 6 muscle (63) on the left arm. After putting the sensor box (10) on the muscle (63), users can start to monitor the information of the muscle (63) following steps S101-S104 of the process of use of the present invention.

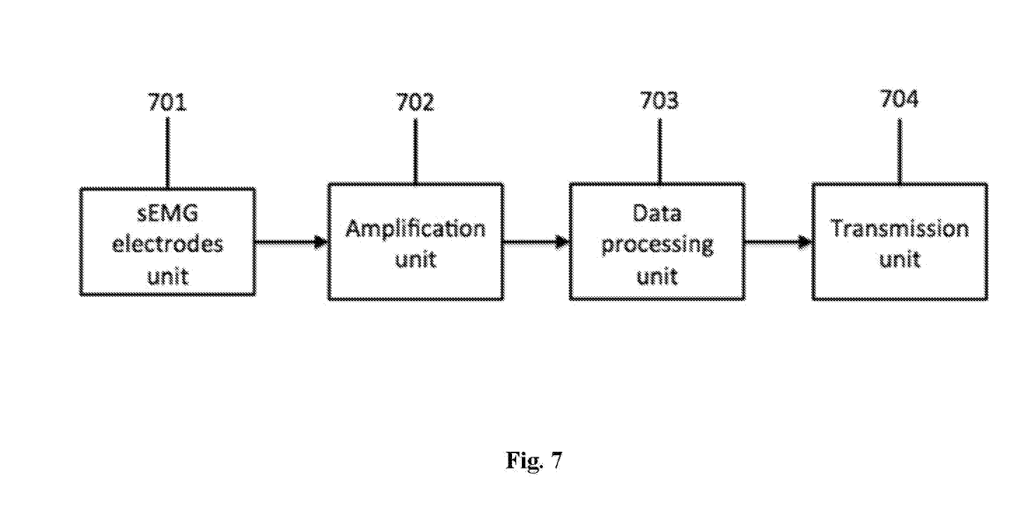

Based on the above system, this invention provides a sEMG home trainer device. As seen in FIG. 7, this device contains at least one sEMG electrodes unit (701), an amplification unit (702), a data processing unit (703) and a transmission unit (704).

The application (or ‘App’) can be pre-loaded on the storage of mobile devices to instruct and guide muscle exercising and rehabilitation functions.

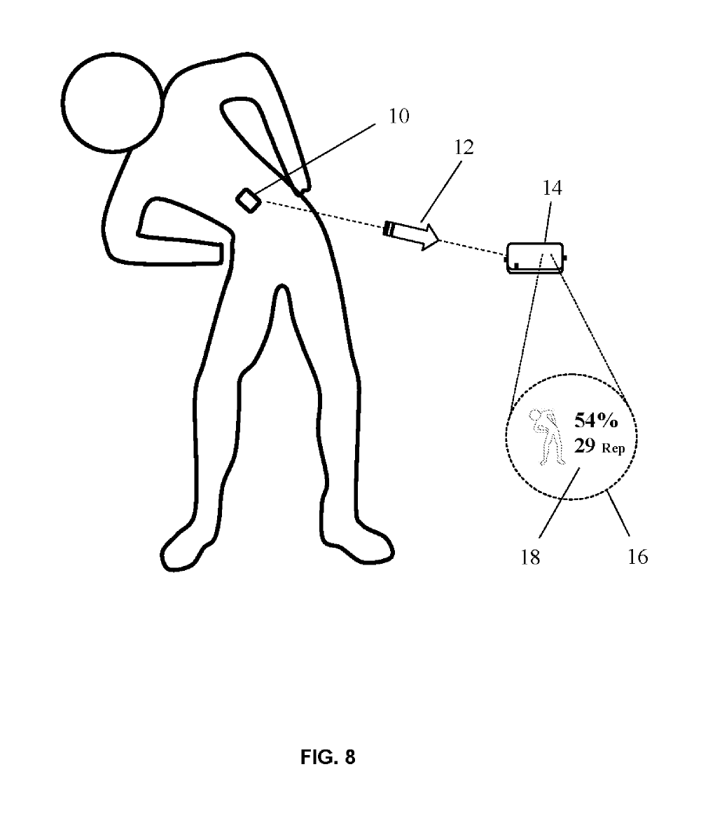

FIG. 8 depicts the preferred embodiment of the present invention shown in use on a user. The sensor box (10), (which includes the at least one sEMG electrodes unit (701), the amplification unit (702), the data processing unit (703), and the transmission unit (704)) is disposed on the chest of the user, and sEMG data is relayed to the mobile device (14) via Bluetooth transfer (12). It is envisioned that, from the mobile device (14), the user will be shown a percentile of muscle stimulation/activation, as well as a counter depicting the number of registered repetitions of muscle contraction shown as data (18). The data (18) is preferably shown on a screen (16) of the mobile device (14), and an audible beep or alarm may be configured to activate after a pre-established number of ‘reps’ have been observed via the present invention. It is envisioned that such an alert would emanate from a speaker of the mobile device (14).

Alternate embodiments of the present invention include variations on the size, color, and shape of the sensor box (10) of the present invention. Similarly, it is envisioned that alternate forms of wireless technology may be employed in lieu of a Bluetooth radio, including WiFi or RF data transfer. The nature of the power source employed within the sensor box (10) is subject to change with advancements in small battery technology.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A system for monitoring muscle activity in real-time during exercise from a mobile device comprising:
a sensor box, said sensor box equipped with a top portion and a bottom portion;
a first silver prong, said first silver prong disposed on said bottom portion of said sensor box;
a second silver prong, said second silver prong disposed on said bottom portion of said sensor box;
a third silver prong, said third saver prong disposed on said bottom portion of said sensor box;
a fourth silver prong, said fourth saver prong disposed on said bottom portion of said sensor box;
a power supply;
wherein said second silver prong and said third silver prong are configured to detect surface EMG potential, producing analog signals;
wherein said first silver prong and said fourth silver prong are grounds;
a pre-amplifier circuit, said pre-amplifier circuit providing a first output of said analog signals;
wherein said pre-amplifier circuit is disposed within said sensor box;
an amplification circuit, said amplification circuit disposed within said sensor box;
wherein said amplification circuit is configured to magnify said first output of said analog signals, providing a second output;
wherein said amplification circuit is configured to filter said analog signals of extraneous noise;
wherein said amplification circuit is configured to convert said analog signals to digital signals;
a DC-offset circuit, said DC-offset circuit configured to off-set the output of
a signal smoothing and amplifier circuit, said signal smoothing and amplifier circuit receiving said pre-amplifier circuit output and said de-offset circuit output;
a CMOS operational amplifier, said CMOS operational amplifier configured to combine said output of said pre-amplifier circuit with said output of said DC-offset circuit to a smoothed and amplified output of EMG data;
a wireless transmitter, said wireless transmitter configured to convey said smoothed and amplified output wirelessly to the mobile device;
an application, said application running on the mobile device; and
wherein said application stores, interprets, and displays said EMG data to the user via a screen of the mobile device.

2. The system of claim 1, wherein said amplification circuit is an AD8220 JFET input instrumentation amplifier.

3. The system of claim 1, wherein said DC-offset circuit employs a REF3012 voltage reference chip.

4. The system of claim 1, wherein said wireless transmitter is a Bluetooth radio.

5. The system of claim 1, wherein said wireless transmitter is a Wi-Fi radio.

6. The system of claim 1, wherein the silver of said first silver prong, said second silver prong, said third silver prong, and said fourth silver prong is of 99.9% purity.

7. The system of claim 4, wherein said Bluetooth radio has a sampling frequency of 1000 Hz.

8. The system of claim 1, wherein the mobile device is an internet-connected smartphone.

9. The system of claim 1, wherein the mobile device is a tablet computer.

10. The system of claim 1, wherein the mobile device is a smartwatch.

11. A method for the collection, magnification, and noise-reduction of sEMG data from a user during exercise comprising:
attaching a sensor box to the skin of the user near a targeted muscle group such that a pair of metal prongs are in contact with the skin of the user;
wherein the sensor box is equipped with an EMG sensor configured to receive EMG potentials from the user via the pair of metal prongs;
collecting EMG data from the skin of the user in the form of analog signals;
processing the analog signals through a pre-amplifier circuit;
the pre-amplifier circuit generating a first output;
shifting the first output from the pre-amplifier circuit with a DC-offset circuit;
filtering the analog signals to delete extraneous noises;
magnifying the analog signals via an amplification circuit, generating a second output;
transferring the analog signals to digital signals;
smoothing the digital signals from the first output with the second output;
relaying the digital signals to a mobile device via a Bluetooth radio;
the mobile device interpreting the digital signals, forming output results;
the mobile device recording the digital signals; and
the mobile device displaying the output results to the user on a screen of the mobile device.
